(19) United States
(12) Patent Application Publication
(10) Pub. No.: US 2022/0379041 A1
(43) Pub. Date: Dec. 1, 2022

(54) DRUG DELIVERY DEVICE WITH A SYSTEM FOR MEASURING A SET DOSE OR A DELIVERED DOSE

(71) Applicant: NEMERA SZCZECIN SP. Z O.O., Szczecin (PL)

(72) Inventors: Daniel Ryszard MATIAS, Szczecin (PL); Alberto LOZANO PLATONOFF, Szczecin (PL)

(21) Appl. No.: 17/770,926

(22) PCT Filed: Oct. 29, 2020
(86) PCT No.: PCT/IB2020/060166

§ 371 Date: Apr. 21, 2022

(30) Foreign Application Priority Data
Oct. 31, 2019 (PL) PL431693

(51) Int. Cl. A61M 5/315

(52) U.S. Cl. CPC A61M 5/31568; A61M 5/31581; A61M 5/31533; A61M 2205/3317; A61M 2205/3561; A61M 2205/502; A61M 5/20

(57) ABSTRACT

A drug delivery device comprising a system for measuring a set dose or a delivered dose comprising at least one magnetic sensor and a magnetic field source characterizes in that the device comprises an element carrying the magnetic field source (9) configured so that during delivering a dose the element moves axially with respect to an element carrying the at least one magnetic sensor (10) measuring a magnetic field, wherein the system for measuring a set dose or a delivered dose comprises a device (13) configured to determine a set dose or a delivered dose based on the magnetic field measurements.

### BACKGROUND

Drug delivery device with a system for measuring a set dose or a delivered dose, in particular a drug delivery device for subcutaneous injection, preferably multiple use injection device.

Monitoring a therapy comprising recording a data about a delivered drug, in particular a dose and a time when it has been delivered, can provide information relevant for a therapy, in particular treatment of chronic disease. This is of particular importance for the patients who are treated by a continuous drug therapy. For example a set of information about the insulin doses delivered including amount of insulin and time of delivery is a part of a self-control as well as an important information for the physicians.

The drug delivery devices for a self-administration of medicament by the patient are used in a treatment of many diseases, in particular in a treatment of the chronic diseases. A dose may be set by a patient before an injection or it may be predetermined in the fixed dose devices. If a dose can be set, its value may be indicated by a setting mechanism, for example as a numerical value appearing in an indication window disposed in a housing of the device. A significant improvement of these devices is a system for measuring a dose set by the setting mechanism or a delivered dose and allowing for recording of this information in an electronic form. This further allows for displaying a delivered dose on a dedicated element of the device, for example LCD (liquid-crystal-display) and/or storing it for later processing or sending to the other devices.

### PRIOR ART

The devices for monitoring operation of a drug delivery devices enabling measurement of a set dose and/or a delivered dose are known in the prior art. In order to detect a dose, usually a displacement of an element of an injection device is measured during dose setting and possible dose correcting or during drug delivery.

Patent application WO2016198516A1 discloses a data collection apparatus adapted for mounting on a drug delivery device and comprising two parts rotatable with respect to each other, wherein one of said parts is rotationally coupled with a dose set knob. Measurement of an amount of relative rotation between said parts provides an information about a delivered dose of drug, wherein the measurement may be a magnetic measurement.

Patent application EP3386566A1 discloses a device for transmitting data from a drug delivery device, which comprises at least one system for detection of rotational movement configured to detect a rotation of at least one physical part of the drug delivery device. A magnet attached to a rotating part of the drug delivery device is used for detecting rotational movement. The magnet together with an electromagnetic switch mounted in the device form a system for detecting data related to a drug delivery, wherein the data are further transmitted by means of a wireless communication.

U.S. Pat. No. 8,632,509B2 reveals an injection device comprising a system enabling measurement of both a set dose and a delivered dose. This is achieved by the independent measurements of a movement of two rotatable elements of this device.

Patent application WO2013120778A1 concerns a measurement system in an add-on device for a drug delivery device. The system comprises two sensors, wherein one of them detects a rotational movement and the other one detects an axial movement by coupling of a sliding element of the measurement system with a dose setting element which is axially movable.

U.S. Pat. No. 7,511,480B2 describes a system for measuring a distance between a housing and a movable sleeve in a drug delivery device with the use of the magneto-resistive sensors utilizing the phenomenon of change of a sensor material resistance in an external magnetic field. The system enables measuring of a set dose and a detection of delivery of the set dose. However it requires a system of the magnetic elements comprising a permanent magnet fixed in the housing and the second magnet with a determined surface profile mounted on a sleeve which is rotationally and axially movable with respect the housing.

The above solutions for measuring a set dose are usually designed for a specific type of a drug delivery device for example the device comprising helically movable sleeve or axially movable activation button for injecting a dose which is arranged at the proximal end of the device. Furthermore in the known solutions the slide elements are used which can reduce their mechanical durability. The another inconvenience of these solutions is an additional resistance during movement of the elements of a system for dose measurement for example by use of the elements coupling with the mechanism of a drug delivery device.

### SUMMARY

The aim of the invention is to provide a drug delivery device with a system for measuring a set dose or a delivered dose providing a precise measurement of a set dose or a delivered dose with a simple and durable construction and without the above inconveniences.

A drug delivery device comprising a system for measuring a set dose or a delivered dose comprising at least one magnetic sensor and a magnetic field source according to the present invention comprises an element carrying the magnetic field source configured so that during delivering a dose the element moves axially with respect to an element carrying the at least one magnetic sensor measuring a magnetic field strength, wherein the system for measuring a set dose or a delivered dose comprises a device configured to determine a set dose or a delivered dose based on the magnetic field measurements.

The element carrying at least one magnetic sensor may be a coupling element, a housing or other element which does not move axially during injection.

Preferably the magnetic field source is a permanent magnet.

Preferably the system for measuring a set dose or a delivered dose comprises at least two, preferably at least four, more preferably at least six magnetic sensors.

Preferably the magnetic sensors are the Hall sensors.

Preferably the magnetic sensors are disposed collinearly.

Preferably the magnetic sensors are disposed at equal linear intervals.

Preferably the magnetic field source is disposed directly on an element configured so that it moves axially with respect to the element carrying at least one magnetic sensor during dose delivery, wherein this element is in a form of a piston rod, a pull-push control nut, a setting sleeve or a dose selector sleeve in non-automatic devices.

Preferably the device comprises a spring element for storing an elastic energy, preferably a helical spring.

Preferably the system for measuring a set dose or a delivered dose comprises a device configured to determine a set dose or a delivered dose based on the magnetic field measurements, preferably in a form of a processor, a microprocessor, a microcomputer or the logic gates system, wherein the device is preferably connected with the sensors by means of a multiplexer.

Preferably the device configured to determine a set dose or a delivered dose based on the magnetic field measurements is connected with an accelerometer.

Preferably the device comprises a display for displaying at least the currently set or delivered dose.

Preferably the device comprises a wireless communication module, preferably Bluetooth module.

Preferably the device comprises a sensor for detecting a dose injection and/or a temperature sensor.

Preferably the device configured to determine a set dose or a delivered dose based on the magnetic field measurements is configured to control the display, the wireless communication module, a sensor for detecting a dose injection and/or temperature sensor.

Construction of a drug delivery device according to the present invention is simple and the device enables a precise measurement of a set dose or a delivered dose without need for using the external devices for example in a form of the dedicated add-ons. Setting a dose may comprise increasing a dose, increasing and resetting a dose or increasing and reducing a dose. Rotation of the magnetic sensors around a magnet does not affect a result of a measurement which depends only on a relative axial position.

A drug delivery device according to the present invention may be also used for measuring a dose delivered by a fixed dose injector allowing for the check of its correct operation.

A drug delivery device according to the present invention may be a disposable device or a reusable device allowing for exchange of a cartridge with medicament.

A drug delivery device according to the present invention may be an automatic applicator, in which at least part of the energy used for expelling a medicament from a cartridge is the energy stored in a spring element. Preferably at least part of the energy is being stored in the spring element during dose setting. Preferably the spring element is a helical spring. During dose setting the spring may be twisted, compressed or twisted and compressed.

Alternatively, in a drug delivery device according to the present invention, the energy used for expelling a medicament from a cartridge is provided directly by a user, in particular by means of pushing a button located on the proximal end of the device.

In a drug delivery device according to the present invention, wherein the device is in a form of an automatic applicator, dose delivery may be activated by pressing a trigger located on a side wall of the housing or pressing a button located on the proximal end of the device.

A device according to the present invention may be used for a delivery of a drug or other liquid preparation by means of a subcutaneous injection, in particular for insulin injection.

### BRIEF DESCRIPTION OF DRAWING

The invention is presented in the exemplary embodiments in the figures, wherein:

FIG. 1 shows a longitudinal cross-section of a drug delivery device with a system for measuring of a delivered dose according to the first embodiment,

FIG. 2 shows a longitudinal cross-section of a drug delivery device with a system for measuring of a set dose or a delivered dose according to the second embodiment,

FIG. 3 shows a longitudinal cross-section of a drug delivery device with a system for measuring of a set dose or a delivered dose according to the third embodiment,

FIG. 4 shows an exemplary block diagram of a system for measuring of a set dose or a delivered dose,

FIG. 5 shows a block diagram of an electronic assembly comprising the system for measuring the dose,

FIG. 6 shows a longitudinal cross-section of a drug delivery device with a system for measuring of a delivered dose according to the sixth embodiment,

FIG. 7 shows a longitudinal cross-section of a drug delivery device with a system for measuring of a set dose or a delivered dose according to the seventh embodiment,

FIG. 8 shows a longitudinal cross-section of a drug delivery device with a system for measuring of a set dose or a delivered dose according to the eighth embodiment,

FIG. 9 shows a dependence of Hall sensor voltage and a magnitude of a set dose with the use of a single magnetic field source in a form of a permanent magnet,

FIG. 10 shows a dependence of Hall sensor voltage and a magnitude of a set dose with the use of two magnetic field sources in a form of a permanent magnet,

FIG. 11 shows a calibration protocol for the system for measuring of a dose,

FIG. 12 shows a protocol of a measurement conducted by the system for measuring of the dose.

### DESCRIPTION OF THE EMBODIMENTS

### Embodiment 1

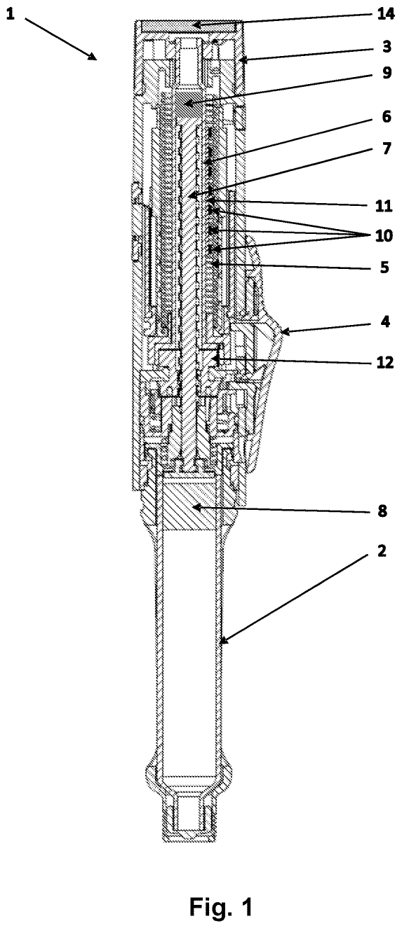

FIG. 1 presents a drug delivery device 1 in a form of an automatic applicator for multiple dosing of the set doses of a medicament from an exchangeable medicament container 2. A knob 3 for setting a dose by a user is disposed at the proximal end of the device, wherein rotation of the knob 3 in a first direction causes increasing a set dose and rotation in a second direction causes reducing a set dose. Dose delivery is activated by axial displacement of a trigger 4 which releases energy stored in a driving spring 5 in a form of a torsion spring mounted loosely on a rotating element 6 (coupling element), which is a rotating sleeve, inside of which a piston rod 7 is located. Simultaneously the piston rod 7 is moved toward the distal end of the device 1 pressing a piston 8 closing the medicament container 2. The energy is stored in the driving spring 5 during increasing a set dose by means of the knob 3. When a dose is reduced part of the energy stored in the driving spring 5 is released but without expelling the medicament from the medicament container 2. When the injection is started by displacing the trigger 4, the medicament is expelled through a needle (not shown in FIG. 1) connecting the medicament container 2 with a surroundings, wherein the needle is mounted on the end of the device, where the medicament container 2 is located.

A system for measuring a dose comprises a magnet 9 disposed on the piston rod 7 pressing the piston 8 closing the medicament container 2 during medicament delivery, seven magnetic sensors 10 in a form of the Hall sensors, disposed colinearly and at equal linear intervals along the piston rod 7. During dose setting the magnet 9 does not move. The magnetic sensors 10 are disposed on a PCB (Printed Circuit Board) 11 fixed to the rotatable element 9 which rotates during dose setting, dose correcting and dose delivery. Rotation of the magnetic sensors 10 around the magnet 9 does not affect the result of a measurement which depends only on their relative axial position.

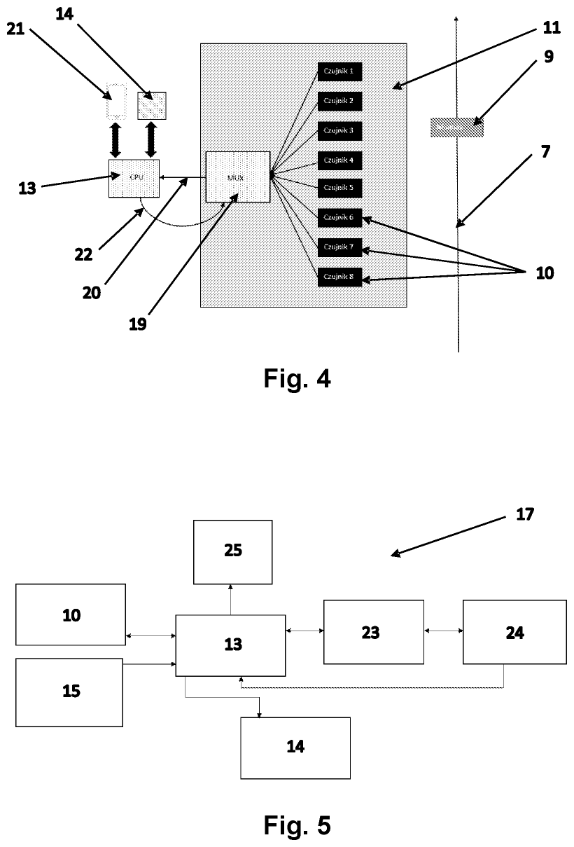

During dose delivery the energy stored in the driving spring 5 causes rotation of a driving nut 12 which drives the piston rod 7 displacing it axially in the distal direction. During its axial movement, the piston rod 7 presses the piston 8 located in the medicament container 2 expelling the medicament from the container. The piston rod 7 is displaced axially by a distance corresponding to amount of the delivered dose. The magnetic sensors 10 measure a strength of a magnetic field generated by the magnet 9 which is displaced axially together with the piston rod 7 by a distance proportional to amount of the delivered dose. The results of these measurements are further converted to information about the dose by a processor 13 (not shown in FIG. 1, shown in FIGS. 4-5) comprised in the system for measuring a dose.

In the depicted embodiment, the system for measuring a set dose or a delivered dose comprises also LCD 14 located in the knob 3. It can show the information about the delivered dose and other information relevant for a user such as time since the last injection or status of a connection with an external device.

An event related to a drug delivery (injection) may be detected by the magnetic sensors 10 sensing change of a magnetic field strength. The device may also comprise an independent system for detecting injection comprising a sensor 15 (not shown in FIG. 1, shown in FIG. 5) which detects injection for example by detection of displacing the trigger 4. A software of the system for measuring a set dose or a delivered dose may also determine and present to a user other information related to a drug delivery such as detecting an injection which probably is a priming of the device (based on an amount of the dose smaller than a threshold value) or sum of the all doses delivered since exchange of the medicament container 2.

All of the magnetic sensors 10 can be identified, hence it is possible to detect movement of the piston rod in both axial directions of the device 1. It is therefore possible for the system for measuring a set dose or a delivered dose to detect that the medicament container 2 has been exchanged because the exchange is related with retracting the piston rod 7 i.e. its displacement in the proximal direction such that it can be contacted with the piston 8 closing the new container 2.

### Embodiment 2

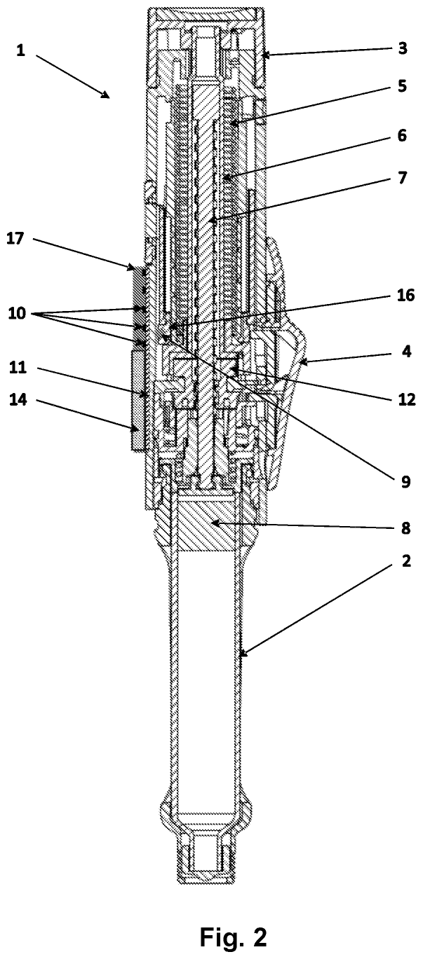

In the embodiment shown in FIG. 2 the device 1 comprises the system for measuring a dose comprising the elements as in the first embodiment, wherein the magnet 9 is attached to a pull-push nut 16 being part of a mechanism indicating a currently set dose of drug. During dose setting the magnet 9 moves axially with the pull-push nut 16, wherein its displacement in a first direction corresponds to increasing a dose and displacement in a second direction corresponds to reducing a dose. The magnetic sensors 10 together with PCB 11 are fixed to a housing of the device 1 and they are located inside an assembly 17 attached to the external surface of the housing. The magnetic sensors 10 measure a strength of a magnetic field generated by the magnet 9 which is displaced axially together with the pull-push nut 16 by a distance proportional to amount of a set dose. The results of these measurements are further converted to information about the currently set dose by the processor 13 (not shown in FIG. 2, shown in FIGS. 4-5).

During dose delivery the energy stored in the driving spring 5 causes rotation of a driving nut 12 which drives the piston rod 7 displacing it axially in the distal direction. During its axial movement, the piston rod 7 presses the piston 8 located in the medicament container 2 expelling the medicament from the container, wherein the pull-push nut 16 moves back to its initial position together with a setting mechanism and an indication mechanism. The axial distance travelled by the pull-push nut 16 during dose delivery is proportional to amount of the delivered dose.

In the depicted embodiment, the system for measuring a set dose or a delivered dose comprises also LCD 14 located in the assembly 17. It can show the information about the delivered dose and other information relevant for a user such as time since the last injection or status of a connection with an external device. Furthermore possibility of measuring of a set dose allows for displaying the information what amount of a dose has been set.

An event of delivery of a drug dose can be detected as in the first embodiment.

### Embodiment 3

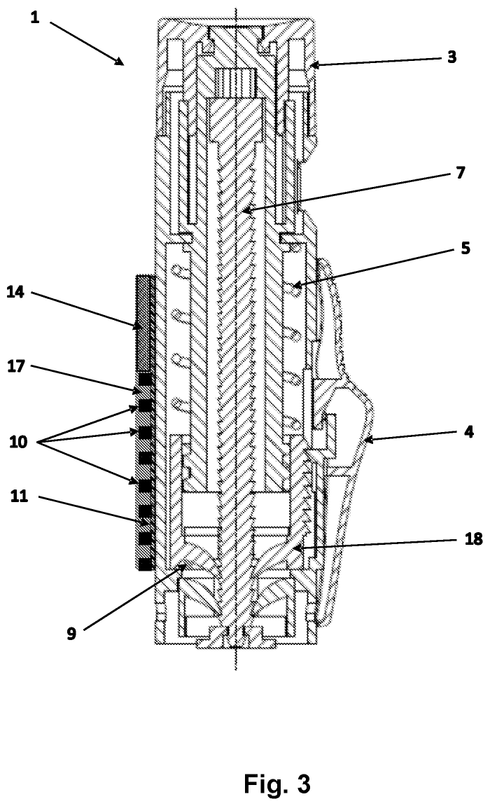

In the embodiment shown in FIG. 3 the device 1 comprises the system for measuring a dose comprising the elements as in the first embodiment, wherein the magnet 9 is attached to a setting sleeve 18 being part of a dose setting and delivering mechanism. When a currently set dose is being increased the setting sleeve 18 moves axially in the proximal direction and when a currently set dose is being reduced the setting sleeve moves axially in the distal direction. During dose delivery the driving spring 5 drives the setting sleeve 18 distally which further drives the piston rod 7 expelling a drug dose. During dose setting the magnet 9 moves axially with the setting sleeve 18. The magnetic sensors 10 together with PCB 11 are fixed to a housing of the measurement assembly 17 located outside the housing of the device 1. The magnetic sensors 10 measure a strength of a magnetic field generated by the magnet 9 which is displaced axially together with the setting sleeve by a distance proportional to amount of a set dose. The results of these measurements are further converted to information about the currently set dose by the processor 13 (not shown in FIG. 3 shown in FIGS. 4-5).

During dose delivery the energy stored in the driving spring 5 causes axial displacement of the setting sleeve 18 in the distal direction and its movement drives the piston rod 7 causing its axial movement in the same direction. During its axial movement, the piston rod 7 presses the piston 8 located in the medicament container 2 expelling the medicament from the container, wherein the setting sleeve 18 moves back to its initial position together with a setting mechanism. The axial distance travelled by the setting sleeve during dose delivery is proportional to amount of the delivered dose.

An event of delivery of a drug dose can be detected as in the first embodiment.

### Embodiment 4

FIG. 4 presents an exemplary block diagram of the system for measuring a set dose or a delivered dose used in the drug delivery device according to the present invention. The system comprises the magnet 9 attached to the axially movable piston rod 7 of the drug delivery device 1, eight magnetic sensors 10, the multiplexer 19, the processor 13 and analog-to-digital converter 20. The magnetic sensors 10 are disposed on the PCB 11 as well as the multiplexer 19, which transfers a specific value of sensors 10 output to the processor 13 by means of the analog-to-digital converter 20 which convert an analog signal into a digital signal. The processor 13 converts the obtained data to the information about a set dose or a delivered dose. It may also record the information in a memory or transfer the information to an external device, in particular a mobile device 21 or display 14. The processor 13 controls also operation of the magnetic sensors 10 by means of the multiplexer 19. This dependency has a reference number 22. Other configurations of an electronic system for converting the data from the magnetic sensors 10 are also possible, for example a result of the measurement may be converted to the information about a set dose or a delivered dose by the drug delivery device 1.

### Embodiment 5

FIG. 5 presents an exemplary block diagram of the assembly 17 comprising the system for measuring a set dose or a dose delivered by the drug delivery device according to the present invention. The assembly 17 comprises the microprocessor 13 controlling its operation, the microprocessor may be disposed on PCB 11 and powered by an energy source (not shown) which may be a battery, in particular rechargeable battery. The microprocessor 13 controls operation of the magnetic sensors 10 comprised in the system for measuring a dose, receives and converts the data from the sensors 10, in particular calculates the amount of a dose based on the magnetic field measurements. In this embodiment the separate sensor 15 is used for detecting injection, for example by detecting an axial displacement of the trigger 4, the sensor 15 may also be a magnetic sensor. The microprocessor 13 controls also a temperature sensor 23 and receives and converts data gathered by this element. The data can be recorded in a memory of the microprocessor 13 or displayed on the display 14. The information about a minimal and maximal ambient temperature to which the drug delivery device 1 has been exposed for a specific period can also be obtained from said data. The microprocessor 13 is also connected with an accelerometer 24 which may be used for awaking the system from a sleep mode and for checking whether the drug delivery device 1 has been exposed to acceleration higher than a threshold value. It enables identifying of the adverse events such that falling of the drug delivery device 1 and warning a user. The microprocessor 13 may control transmitting data to the external device 21 by means of a wireless communication means 25, in particular Bluetooth module and displaying data on the screen of the display 14.

Besides the described exemplary embodiments of the system for measuring a dose, the other specific means known in the art may be used. Other means may be used in particular in the field of the peripheral devices for data transmitting and displaying.

### Embodiment 6

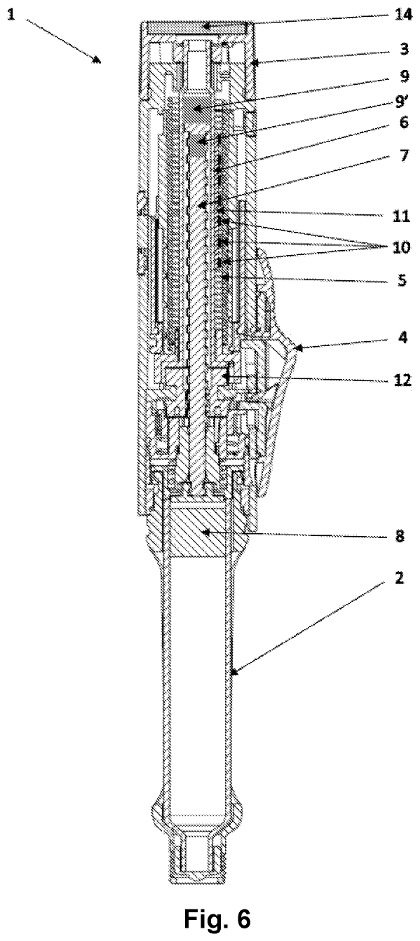

In the embodiment shown in FIG. 6 the drug delivery device 1 comprises the system for measuring a dose comprising the elements as in the first embodiment, wherein the system comprises an additional magnet 9′ which is disposed on the piston rod 7 pressing the piston 8 closing the medicament container 2 during medicament delivery. The additional magnet 9′ is disposed at such axial distance from the magnet 9 that it is possible to detect phase shift between the magnetic field generated by the magnet 9 and the additional magnet 9′ by means of the magnetic sensors 10. The magnets 9 and 9′ are identical.

A place where the additional magnet 9′ is attached to the piston rod 7 may be different than in this embodiment. The magnet 9′ may be attached to the piston rod 7 or it may be disposed inside the piston rod 7. The magnet 9′ may be also disposed around the piston rod 7 or a part of the piston rod may be made from a magnetic material. All alternative locations of the additional magnet 9′ applies also to the magnet 9.

### Embodiment 7

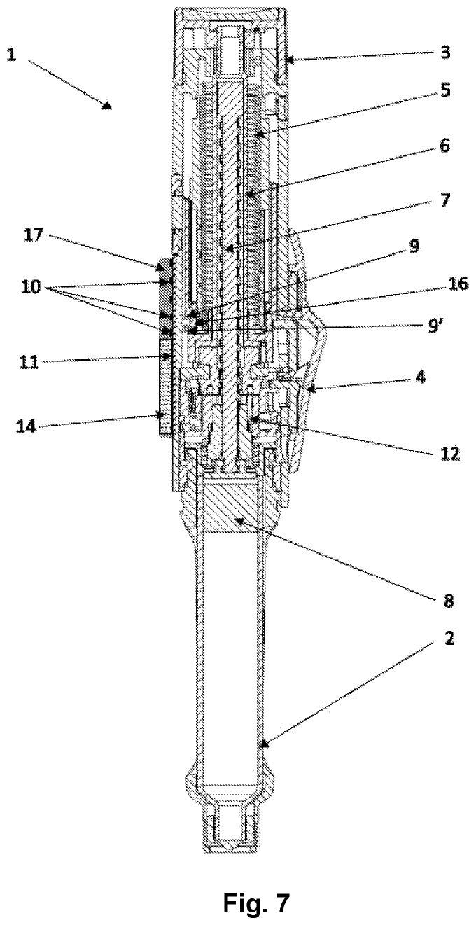

In the embodiment shown in FIG. 7 the drug delivery device 1 comprises the system for measuring a dose comprising the elements as in the second embodiment, wherein the system comprises an additional magnet 9′ which is attached to the pull-push nut 16 being part of a mechanism indicating a currently set dose of drug. The additional magnet 9′ is disposed at such axial distance from the magnet 9 that it is possible to detect phase shift between the magnetic field generated by the magnet 9 and the additional magnet 9′ by means of the magnetic sensors 10. The magnets 9 and 9′ are identical.

### Embodiment 8

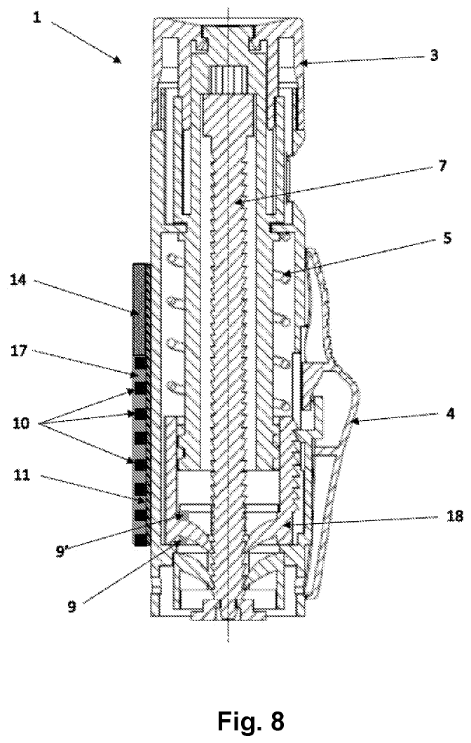

In the embodiment shown in FIG. 8 the drug delivery device 1 comprises the system for measuring a dose comprising the elements as in the third embodiment, wherein the system comprises an additional magnet 9′ which is attached to the setting sleeve 18 being part of a dose setting and delivering mechanism. The additional magnet 9′ is disposed at such axial distance from the magnet 9 that it is possible to detect phase shift between the magnetic field generated by the magnet 9 and the additional magnet 9′ by means of the magnetic sensors 10. The magnets 9 and 9′ are identical.

### The Additional Features of the System for Measuring a Set Dose or a Delivered Dose and the Electronic Assembly.

The system for measuring a set dose or a delivered dose presented as the embodiment 4 and in FIG. 4 may be used in all implementations of the device 1 such as the embodiments 1-3 and 6-8 and other devices not mentioned explicitly. The same applies to the electronic assembly comprising the system for measuring a dose presented as the embodiment 5 and in FIG. 5.

In the embodiments according to the embodiments 6, 7 and 8 a magnetic field source is more than one magnet. In these embodiments there are two identical magnets—the magnet 9 and the additional magnet 9′ attached to the same element of the device and shifted with respect to each other in the axial direction. Alternatively the magnets may be attached to different axially movable elements or two elements coupled axially. The magnets 9 and 9′ may not be identical. The system for measuring a dose may comprise more than one additional magnet 9′, preferably two, three or four additional magnets.

The magnets 9 and 9′ generate the same magnetic field but due to a specific distance between the magnets their contributions to the magnetic field, detected by the magnetic sensors 10, are phase-shifted. This allows for achieving better precision of measuring a position of the element carrying a magnetic field source. It also enhances a reliability of the system for measuring a dose, in particular allows for reducing adverse effects of inaccuracies in mechanical system of the drug delivery device manufacturing and, as a result, broadens a scope of the possible implementations.

Additional preferable feature enhancing measurement precision is disposing the magnetic sensors 10 such that the element carrying a magnetic field source is within their range in all possible positions of this element during operation of the device.

The most preferable relative position of the magnets 9 and 9′ depend on many factors such that strength of the magnets, accuracy and position of the magnetic sensors 10 and working range of the element carrying the sensors. The magnets should be spaced so that the magnetic sensors 10 are able to detect a phase shift between the magnetic field generated by the magnets. But if the distance between the magnets is too long, it can adversely affect a size of the system and possible applications in the handy drug delivery devices.

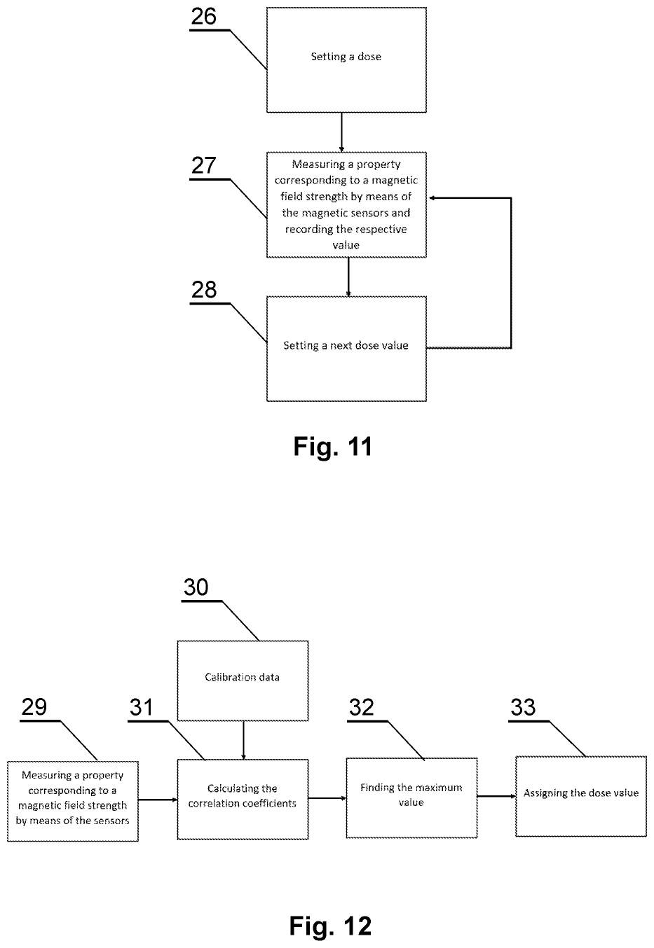

A transverse distance between the magnet (or magnets) and the magnetic sensors 10 may also play a role in practical application, independently of a number of the magnets. It should be selected so that a detection signal is not too weak and so that the magnetic sensors are not saturated due to too high values of a magnetic field strength. Hence too small or too large transverse distance may adversely affect measurement precision. A suitable relative position of the magnets may be determined so that it minimizes inaccuracy of a measurement made according to a procedure shown in FIGS. 11 and 12.

The Characteristic Features of a Signal from the Magnetic Sensors of the Dose Measurement System

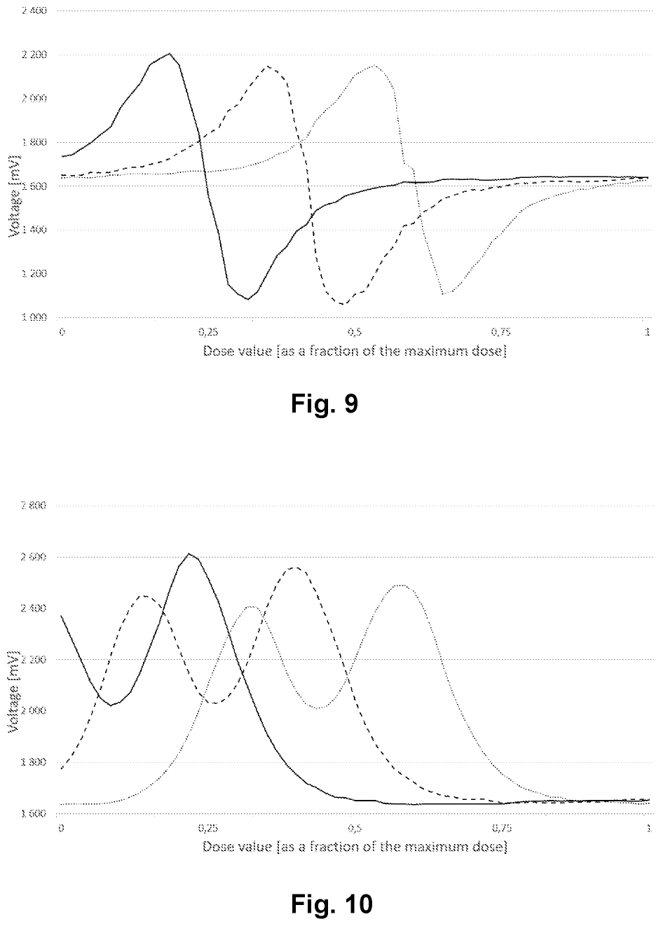

FIG. 9 presents dependence of a voltage of the magnetic sensor 10 in a form of Hall sensor and a magnitude of a set dose with the use of a single magnetic field source in a form of the magnet 9. The respective curves in the figure depict voltage for three, equally spaced, subsequent magnetic sensors 10 in a form of the Hall sensors. The dependence is presented with respect to the maximum dose. The figure reflects the shapes of the curves for the embodiments 1, 2 and 3.

FIG. 10 presents dependence of a voltage of the magnetic sensor 10 in a form of Hall sensor and a magnitude of a set dose with the use of two magnetic field sources in a form of the permanent magnets. The respective curves in the figure depict voltage for three, equally spaced, subsequent magnetic sensors 10 in a form of the Hall sensors. The dependence is presented with respect to the maximum dose. The figure reflects the shapes of the curves for the embodiments 6, 7 and 8.

### Exemplary Calibration Protocol for the Dose Detection System

FIG. 11 shows schematically calibration protocol for the dose detection system. The calibration begins with setting a dose and further measurement of a property corresponding to a magnetic field strength by means of the magnetic sensors 10 and recording the respective value 27. Then a next dose value 28 is set. The steps 27 and 28 are repeated for all possible doses. The result of the calibration is a calibration matrix 30 including the values of a property corresponding to a magnetic field strength for the specific sensors and the specific dose magnitudes. A property corresponding to a magnetic field strength is usually a voltage. It may also be another electric property or any other property varying with the changes in a magnetic field strength detected by the sensors, preferably proportional to a magnetic field strength. An exemplary calibration protocol may be used in all dose detection systems described as the embodiments.

### Exemplary Measurement Protocol

FIG. 12 shows schematically measurement protocol realized by the dose measurement system. At the beginning a property corresponding to a magnetic field strength is measured by means of the sensors 29 and the correlation coefficients between a set of values measured at the first step and the data from calibration matrix are calculated 31. Then a maximum correlation coefficient is determined 32 and a measured dose is the dose corresponding to this maximum coefficient. An exemplary measurement protocol may be used in all dose detection systems described as the embodiments. A property corresponding to a magnetic field strength is usually a voltage. It may also be another electric property or any other property varying with the changes in a magnetic field strength detected by the sensors, preferably proportional to a magnetic field strength.

1. A drug delivery device comprising a system for measuring a set dose or a delivered dose comprising at least one magnetic sensor and a magnetic field source, wherein the drug delivery device comprises an element carrying the magnetic field source configured so that during dose setting and delivering a dose said element moves axially with respect to an element carrying the at least one magnetic sensor measuring a magnetic field strength, wherein the system for measuring a set dose or a delivered dose comprises a device configured to determine a set dose or a delivered dose based on the magnetic field measurements, a sensor for detecting a dose injection and a spring element for storing an elastic energy for the automatic injection of the set dose.

2. The device of claim 1, wherein the magnetic field source is a permanent magnet.

3. The device of claim 1, wherein the system for measuring a set dose or a delivered dose comprises at least two sensors.

4. The device of claim 3, wherein the magnetic sensors are the Hall sensors.

5. The device of claim 3, wherein the magnetic sensors are disposed collinearly.

6. The device of claim 5, wherein the magnetic sensors are disposed at equal linear intervals.

7. The device of claim 1, wherein the magnetic field source is disposed on an element configured so that it moves axially with respect to the element carrying at least one magnetic sensor, wherein this element is a piston rod, a pull-push control nut, a setting sleeve or a dose selector sleeve.

8. The device of claim 1, wherein the spring element is a helical spring.

9. The device of claim 1, wherein the device configured to determine a set dose or a delivered dose based on the magnetic field measurements is a processor, a microprocessor, a microcomputer or a logic gates system.

10. The device of claim 1, wherein the device configured to determine a set dose or a delivered dose based on the magnetic field measurements is connected with the sensors by means of a multiplexer.

11. The device of claim 1, wherein the device configured to determine a set dose or a delivered dose based on the magnetic field measurements is connected with an accelerometer.

12. The device of claim 1, wherein the device comprises a display for displaying at least the currently set or delivered dose.

13. The device of claim 1, wherein the device comprises a wireless communication module.

14-15. (canceled)

16. The device of claim 13, wherein the communication module is the Bluetooth module.

17. The device of claim 1, wherein the device comprises a temperature sensor.

18. The device of claim 1, wherein the device configured to determine a set dose or a delivered dose based on the magnetic field measurements is configured to control the display, the wireless communication module, a sensor for detecting a dose injection and/or temperature sensor.
